(19) United States
(12) Patent Application Publication
(10) Pub. No.: US 2023/0407237 A1
(43) Pub. Date: Dec. 21, 2023

(54) MUTANT STRAIN OF THE ALGA NANNOCHLOROPSIS AND METHOD OF PRODUCTION OF THE SAME, ITS USE IN THE PRODUCTION OF ASTAXANTHIN AND OMEGA-3 AND RELATED COMPOSITIONS

(71) Applicant: UNIVERSITÀ DEGLI STUDI DI VERONA, Verona (IT)

(72) Inventors: Matteo BALLOTTARI, Verona (IT); Michela CECCHIN, Verona (IT); Stefano CAZZANIGA, Verona (IT); Stefania PALTRINIERI, Verona (IT)

(21) Appl. No.: 18/251,498

(22) PCT Filed: Oct. 28, 2021
(86) PCT No.: PCT/IB2021/059969

§ 371 Date: May 2, 2023

(30) Foreign Application Priority Data
Nov. 2, 2020 (IT) 102020000026005

(51) Int. Cl. C12N 1/12; C12P 7/6432; C12P 23/00

(52) U.S. Cl. CPC C12N 1/125; C12P 7/6432; C12P 23/00; C12R 2001/89

(57) ABSTRACT

The mutant strain of the alga Nannochloropsis is provided. A method for producing the mutant strain of the alga Nannochloropsis is also provided. Use of compounds produced from the mutant strain of the alga Nannochloropsis is further provided.

Microalgae are photoautotrophic agents that may be grown to produce biomass and high-energy and/or high-value products.

Some of these organisms are in fact able to produce and accumulate high amounts of, for example, carotenoids and/or lipids, which are used and/or usable as food additives or for the production of biofuels.

Among the main high-value products produced by microalgae, the products called Astaxanthin (ASX) and Omega-3 (EPA) have been of particular interest.

Astaxanthin is a commercially valuable carotenoid produced by various engineered microalgae and/or microorganisms such as, for example, bacteria and yeast.

Carotenoids are biological compounds involved in many protective mechanisms, derived from microalgae and plants and useful for human health, because they have, among other things, a significant antioxidant activity, essential to avoid the harmful effects of free radicals.

Diets rich in carotenoids protect against several diseases, such as cancer, cardiovascular disease, and arthritis, and may improve the health of patients with AIDS, diabetes, macular degeneration, and neurodegeneration.

Due to their properties, they have gained enormous commercial value in recent years; the most valuable ones are 3-Carotene and Astaxanthin, which cover more than half of the current carotenoid market.

Astaxanthin shows the highest antioxidant capacities, e.g., 44%-600% higher than vitamin E and 3-carotene, respectively, and has been shown to be completely safe, while 3-carotene has been described as carcinogenic with prolonged/excessive intake.

For this reason, Astaxanthin represents the best candidate for commercial uses, such as in food and/or feed supplements, in cosmetics, or combined directly with pharmaceutical agents in preventive therapies.

Astaxanthin is a carotenoid used primarily in dietary supplementation and as a pigmenting agent in aquaculture.

Synthetic Astaxanthin (which accounts for 95% of the market) is produced from petrochemical sources, creating, however, problems of potential toxicity and pollution, and thus raising questions of environmental sustainability.

These problems are increasingly directing the research efforts of those skilled in the art toward a production of Astaxanthin from microalgae (e.g., Haematococcus pluvialis and/or Chlorella zofingensis), but current approaches still have significant drawbacks.

Despite high levels of accumulation achieved through the cultivation of H. pluvialis, the Astaxanthin obtained is disadvantageous due to the high costs encountered during the production, extraction, and purification of this molecule; in fact, the production of Astaxanthin from H. pluvialis currently requires a two-phase cultivation system: in the first phase the so-called “green” biomass is generated, while in the second phase the biosynthesis of Astaxanthin is induced by stressing the cell culture, e.g., through high light intensity, nutrient depletion, and other stress conditions commonly known in the art.

Furthermore, the cell wall of this microalgae species is composed of a trilaminar sheet, which requires complex and expensive destructive methods for its degradation.

Recent large-scale studies have calculated that these production costs amount to about €1,500/kg in the best case; moreover, the presence of rigid cell walls negatively affects yield, quality, and bioavailability of recovered bioactive compounds.

Consequently, it is not surprising that most of the Astaxanthin on the market is produced synthetically, at a cost of about €880/kg, while Astaxanthin derived from H. pluvialis corresponds to only <1% of the amount sold.

On the other hand, synthetic Astaxanthin has antioxidant properties far inferior to natural Astaxanthin (for example, the natural one is 20 times more powerful in eliminating free radicals) and has not been approved for human consumption by the FDA (Food and Drug Administration, USA).

Alternative production methods have attempted to overcome these limitations by starting with different microalgae species, but a commercially viable system has yet to be implemented.

In addition to H. pluvialis, a few algal species may produce Astaxanthin at detectable levels, such as, for example, C. zofingensis and C. nivalis; however, industrial-scale cultivation of these algal strains for Astaxanthin accumulation is not sustainable, due to both low production yields and the presence of robust cell walls, which impose expensive and detrimental extraction methods for the desired substances.

Omega-3s (ω-3) are long-chain fatty acids, essential nutrients for vertebrates.

In humans, they help maintain cell membranes, brain function, and nerve impulse transmission under normal conditions.

Omega-3s also exert a key role in the processes of oxygen transfer to blood plasma, hemoglobin synthesis, and cell division.

They are also indicated for the prevention and/or treatment of cardiovascular disease and in neurological treatments by improving concentration, memory, motivation, and motor skills, as well as preventing degenerative brain diseases.

In pregnancy, they reduce the risk of postpartum depression and mood swings.

Although Omega-3s are primarily produced from marine microalgae, current production methodologies rely on their extraction from fish or krill oils due to lower production costs.

Algae species belonging to the genus Nannochloropsis are considered among the most interesting unicellular marine microalgae (Hibberd, 1981) for large-scale cultivation, both in open ponds and in closed systems, and may be considered good candidates for biodiesel production due to their high growth rate (Sforza et al., 2010), high lipid accumulation (up to 65-70% of total dry weight), and ability to adapt to different types of irradiations (Boussiba et al., 1987, Hodgson et al., 1991, Rodolfi et al., 2008).

In addition, the fatty acids found in Nannochloropsis are composed of 35% polyunsaturated fatty acids (so-called PUFAs, specifically, eicosapentaenoic acid (EPA, 20:5 ω3)), which are compounds of high nutritional value for human health (Gill and Valivety 1997).

For these reasons, the genus Nannochloropsis is an industrially promising candidate as a platform for the production of EPA for human use.

However, its use, in particular, the use of the species Nannochloropsis gaditana, for the production of EPA is not available industrially due to the high costs associated with the cultivation of microalgae.

Major sources of natural Astaxanthin (wild-type, W.T.) are crustaceans, yeast, bacteria, and microalgae.

Crustaceans contain appreciable amounts of Astaxanthin (ASX), carotenoids, long-chain fatty acids, and several high-value nutrients.

ASX is obtained from these raw materials by chemical extraction.

Process-related reagents, as well as additives used during the cultivation, harvest, processing, storage, distribution, and consumption of source species, may pose health risks or allergy problems.

Exposure of crustaceans to different habitats may unfortunately be associated with the presence of parasites, biotoxins, bacteria, and heavy metals; moreover, the Astaxanthin content in crustaceans is low compared to other natural sources.

Thus, various reasons make different production methods preferred.

Yeasts, such as, for example, Phaffia rhodozyma, may produce Astaxanthin by biological fermentation.

Phaffia rhodozyma is currently the most widely used yeast species, due to the high yield of the production process.

The yield may be higher than that of other yeasts, but lower than other microorganisms.

A key reason to use Phaffia rhodozyma for Astaxanthin production is offered by the rapid proliferation of this microorganism and the ease of destruction of yeast cells, allowing easy access to the target molecule and efficient isolation.

A relative disadvantage of using this microorganism is that the concentration of the naturally occurring molecule in the microorganism is in any event very low.

Production on a commercial scale is obtained through genetic mutations of the original species, which, however, pose safety and regulatory issues for the introduction of the resulting product into the human food chain.

Therefore, this product is only used as an animal feed supplement.

Astaxanthin may also be produced by some bacteria such as, for example, Paracoccus spp., Agrobacterium spp., Sphingomonas spp., Pseudomonas spp.

Paracoccus carotinifaciens is one of the most studied and used species because it is a bacterium rich in carotenoids.

Overall, it contains a rich mixture of carotenoids, in which ASX predominates significantly by weight (2.2%).

Similar to Phaffia rhodozyma, enhancement of production is achieved by mutagenesis and genetic engineering.

This bacterium mainly finds application in animal feed and is not approved for direct human consumption.

Among the 200,000-800,000 species of algae that exist in nature, only a few are used in food applications because of the stringent requirements for bringing algae derivatives as nutraceutical components to the market.

Haematococcus pluvialis (also known as Haematococcus lacustris), is the most widely used alga for the production of ASX, since it is characterized by a high natural capacity to produce and accumulate Astaxanthin with respect to the dry biomass produced (from about 1.5 up to 5% by weight (w/w)).

In 1991, H. pluvialis was granted GRAS (i.e., Generally Recognized As Safe) status by the Food and Drug Administration (FDA).

In 2017, it was also declared safe for human consumption (at specific daily intake dosages) in Europe.

The structure of Astaxanthin obtained from H. pluvialis is very similar to that obtained from salmon and other aquatic organisms, becoming, therefore, highly absorbable by the human body.

The industrial production of Astaxanthin from H. pluvialis is presently achieved through a two-stage batch method consisting of a first phase, the so-called “green stage,” which usually lasts from 9 to 20 days and corresponds to the growth period of the algal cells under appropriate conditions, and a second phase, the so-called “red stage,” which is usually continued for a period of 6 days, during which the algal cells are subjected to stress conditions that cause the accumulation of Astaxanthin as a defense mechanism.

The productivity of Astaxanthin from H. pluvialis may reach 8-10 mg/L/day in a total cycle of about 10 days (about 4 days in the “green stage” phase and about 6 days in the “red stage” phase) with a percent concentration by weight (w/w) around 4%.

A disadvantage of the “red stage” is that stress factors may potentially lead to cell death, effectively reducing the overall yield of the process; moreover, this method has high production costs due to the high consumption of electricity to provide adequate illumination.

The “red stage,” moreover, also produces mechanically and chemically resistant cell walls, requiring, therefore, complex and expensive procedures for the extraction of the products of interest.

Recently, alternative methods achieving three-stage or single-stage production processes have been proposed.

Single-stage production is achieved by combining the “green” growth phase and the “red” accumulation phase into a single operation; overall, this type of process simplifies plant operations, including a reduction in cost.

However, it was generally less productive than the two-stage batch method; in addition, the total process duration is about 8-11 days.

At present, few types of preparations allow the production of natural Astaxanthin while achieving an efficient production, a short production cycle, high process yields, and compliance with the requirements of regulatory authorities regarding human nutrition.

On the other hand, for Omega-3, its current primary sources are fish oil and fish meal originating from the sea from the aquaculture sector.

Due to the growing consumption of Omega-3 rich oils, there is an increasing deficit in its production, because industries still depend on fish as its main source.

Overfishing, which causes depletion of fish stocks, and heavy metal contamination are also key factors that make this method of production increasingly critical.

The unpleasant taste and smell of the oil, as well as its stability problems, lead to high production costs and limit the market of this product.

Problems also derive from the possible presence of harmful contaminants, such as teratogenic, mutagenic, and carcinogenic agents, but also non-carcinogenic agents, such as antibiotics and heavy metals; moreover, the content of Omega-3 in farmed fish depends essentially on the quantity that the different species take with their diet.

Marine fish oil and fish meal are included in the diets of farmed fish to enhance their Omega-3 content; thus, paradoxically, the aquaculture sector is the main supplier but also the main user of Omega-3 fatty acids.

This fact creates an unsustainable business that ultimately raises a number of ethical questions as well.

Although industry experts expect that both Omega-3 and Astaxanthin may, starting in 2020, be produced primarily from microalgae, despite the large market potential and growing demand for naturally produced high-value food components, microalgae are still far from becoming an economically viable production alternative.

### SUMMARY OF THE INVENTION

The inventors of this patent application have unexpectedly identified a mutant strain of Nannochloropsis gaditana capable of producing high amounts of astaxanthin and Omega-3 at the same time according to a very advantageous process, from an industrial point of view.

### BRIEF DESCRIPTION OF THE DRAWINGS

FIG. 1 shows the copy of the certificate of deposit of the ASTAOMEGA (formerly Nannochloropsis gaditana D23) mutant strain of this invention with the CCAP-SAMS International Depositary Authority.

FIG. 2 shows the list of identified mutations of the ASTAOMEGA mutant strain of this invention.

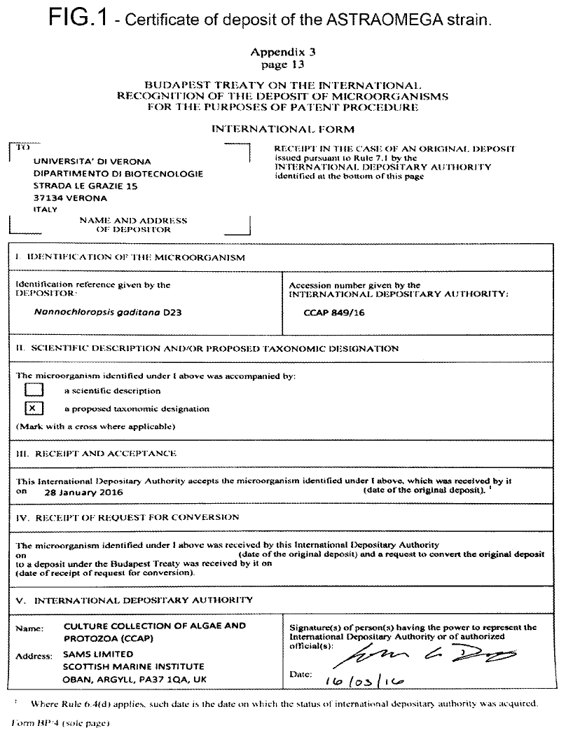

### SUBJECT MATTER OF THE INVENTION

In a first subject matter, this invention describes a mutant strain of the seaweed Nannochloropsis.

In a second subject matter, this invention describes a method for obtaining it.

In a third subject matter, this invention describes a process for the production of Astaxanthin, ketocarotenoids, and Omega-3 (EPA), comprising the use of said mutated strain.

In a fourth subject matter, this invention describes food and nutraceutical compositions comprising compounds produced by the mutated strain.

In other subjects, this invention describes the use of compounds produced from the mutated strain for use in the food supplement and nutraceutical industry, the pharmaceutical and/or cosmetic industry, and the aquaculture industry.

### DETAILED DESCRIPTION OF THE INVENTION

According to a first subject matter, this invention describes a mutant strain of the seaweed Nannochloropsis.

This ASTAOMEGA mutant strain has been created and selected at the Department of Biotechnology of the University of Verona, by the group directed by Prof. Matteo Ballottari.

Said strain has been deposited with the CCAP-SAMS International Depositary Authority (CULTURE COLLECTION OF ALGAE AND PROTOZOA (CCAP)—SAMS Limited Scottish Marine 18 Institute, OBAN, Argyll, PA37 1QA, UK) on Jan. 28, 2016, and registered under CCAP Access Number 849/16 (the name indicated Nannochloropsis gaditana D23 was the identifying abbreviation initially assigned by the authors to the strain, later changed by said authors to ASTAOMEGA, as used for convenience in this description).

According to a second subject matter, this invention describes a method for obtaining the aforementioned mutated strain.

In particular, this method comprises the step of random chemical mutagenesis carried out by exposing N. gaditana W.T. (strain obtained from the CCAP-SANS Institute, Access Number CCAP849/5) to a mutagenic agent represented by EMS (ethyl methanesulfonate or ethyl mesylate; Merck Index, 11th Ed, 3782) following the procedure described in Cecchin et al 2020 (Improved lipid productivity in Nannochloropsis gaditana in nitrogen-replete conditions by selection of pale green mutants, Cecchin M, Berteotti S, Paltrinieri S, Vigilante I, Iadarola B, Giovannone B, Maffei M E, Delledonne M, Ballottari M. Biotechnol Biofuels. 2020 Apr. 21; 13:78. doi: 10.1186/s13068-020-01718-8. eCollection 2020, which is incorporated herein in its entirety as reference).

Specifically, the EMS compound was added to 10⁸ cells/mL at final weight/volume percentages of 0.75%, 1.5%, 2%, and 2.5%.

Samples were incubated for 2 hours in the dark and then diluted in 10% sodium thiosulfate solution to inactivate the mutagen activity.

The cells were then centrifuged at 6000 g, washed twice with 1 M NaCl, dissolved in 500 μl of f/2 growth medium (commercially available), and maintained overnight under low light conditions.

The cells were then plated on solid f/2 medium and kept under low-light conditions (50 μmol m⁻² s⁻¹) for at least 2 weeks.

The cells treated with EMS concentrations that induce 95% mortality (determined as the number of colonies on plate in the EMS-treated cells compared with the number of colonies on plate of the sample not exposed to the mutagen) were used for the subsequent screening procedure.

This concentration was found to be 2% EMS.

The EMS treatment generated variants in the genome of early N. gaditana W.T. creating a library of mutants.

The different strains obtained from single colony on plate were classified and selected according to the different pigment composition.

Specifically, strains with different carotenoid/chlorophyll ratios were selected and further characterized based on the 500/680 nm absorption ratio of the total pigments extracted.

The ASTAOMEGA CCAP 849/16 mutant (formerly Nannochloropsis gaditana D23) was particularly notable for having a high 500/680 nm ratio, due to a high carotenoid/chlorophyll ratio, with an accumulation of Astaxanthin up to 1% of its dry weight, as subsequently verified by HPLC.

According to a particular aspect of this invention, the carotenoid/chlorophyll ratio is increased up to 150% with respect to the wild-type strain.

The characterization of the ASTAOMEGA genotype by whole-genome sequencing revealed the presence of 504 mutations.

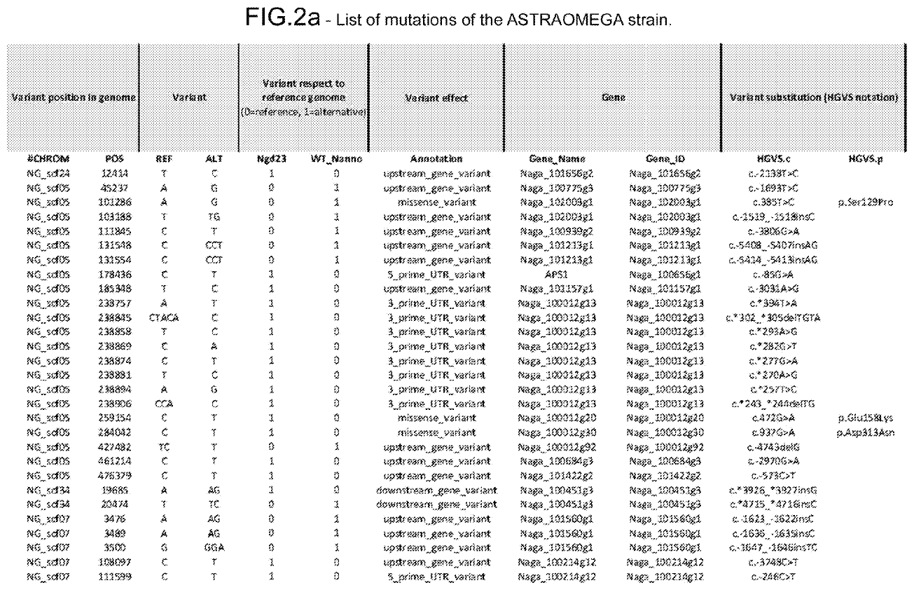

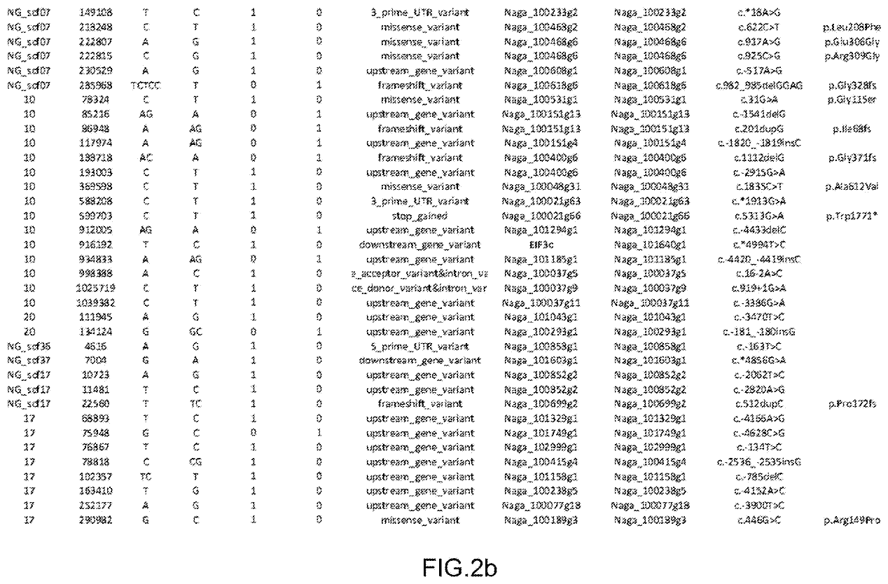

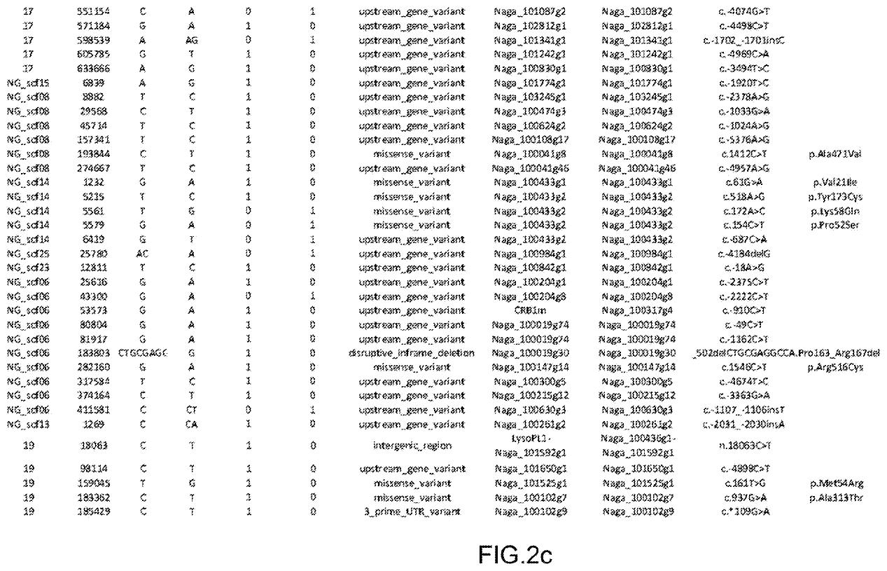

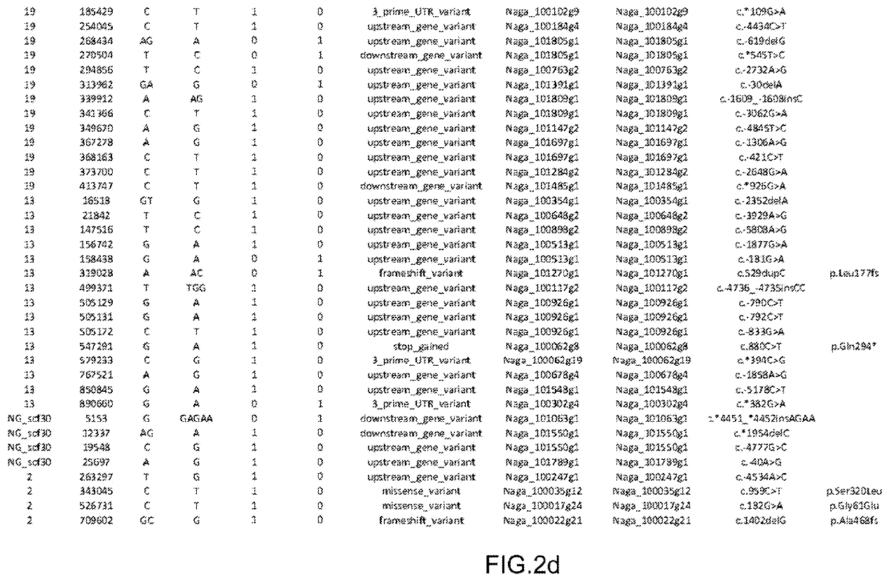

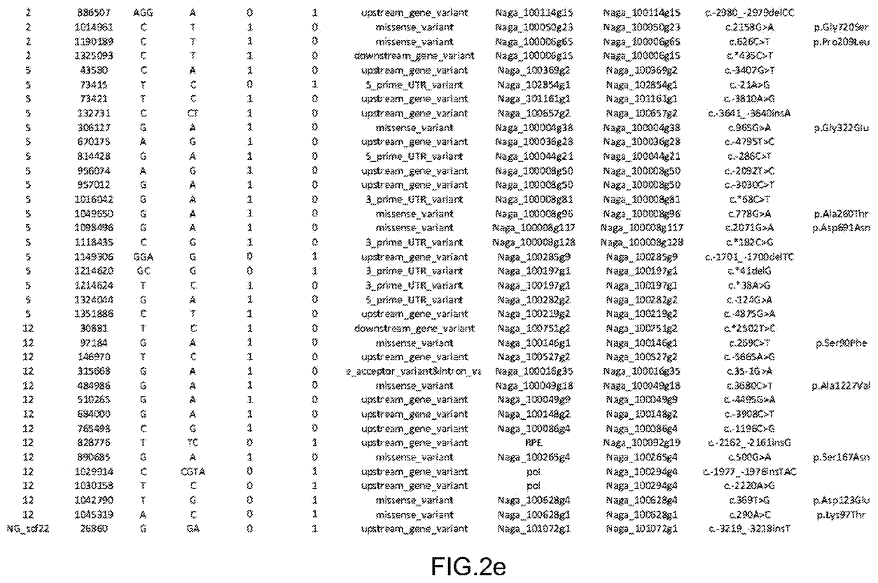

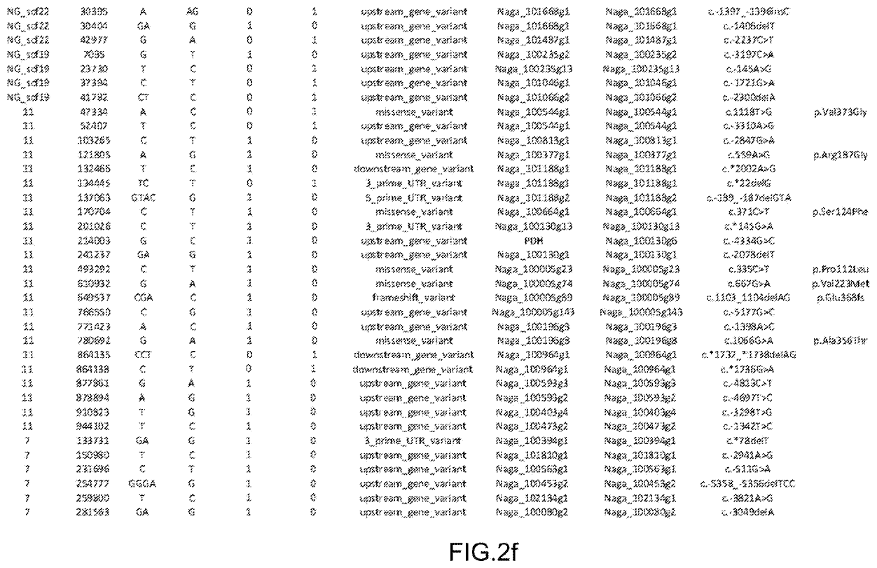

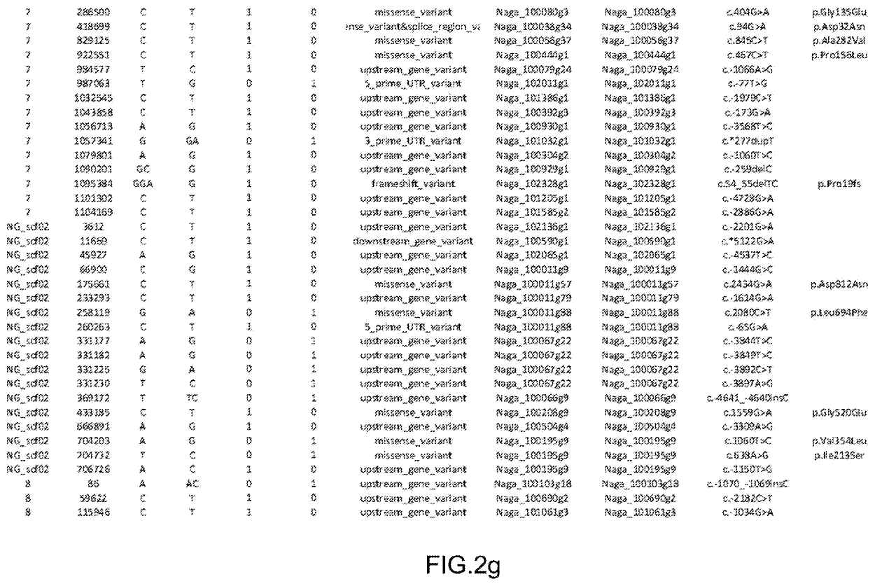

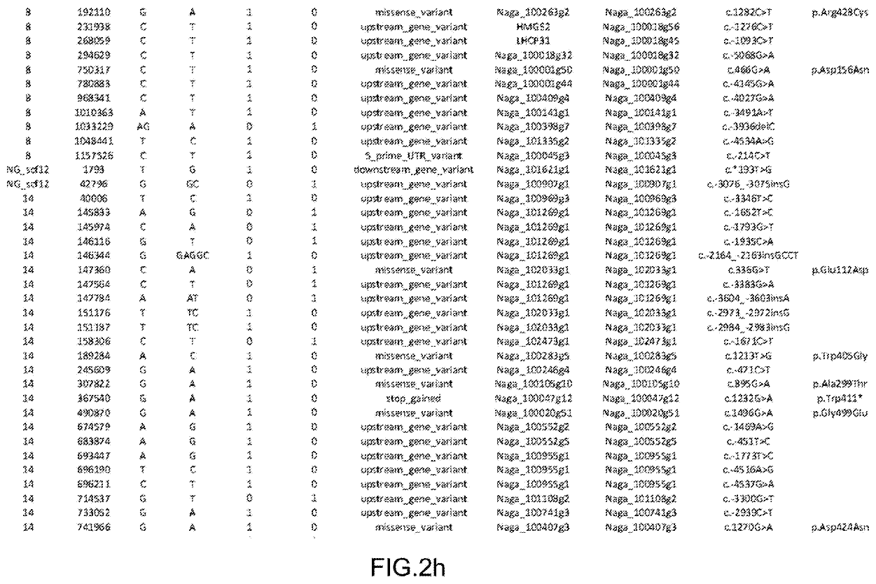

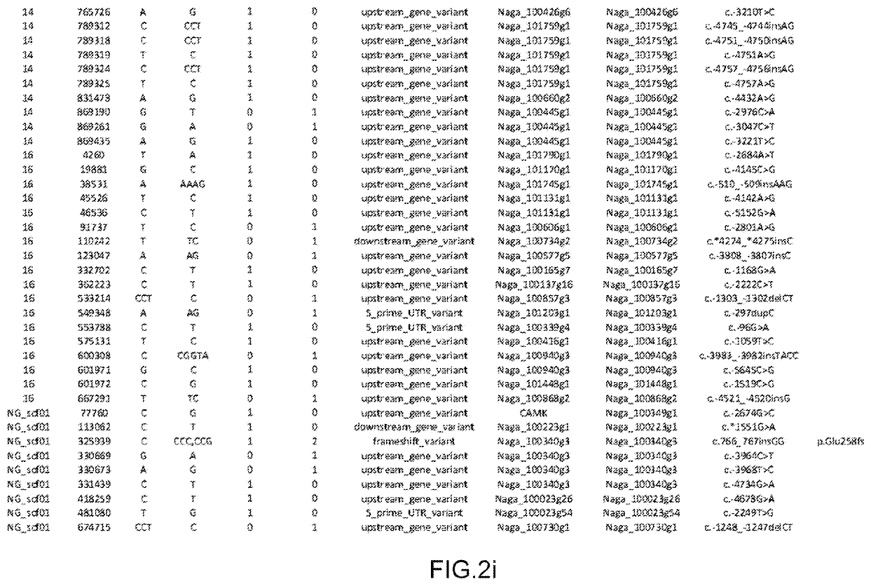

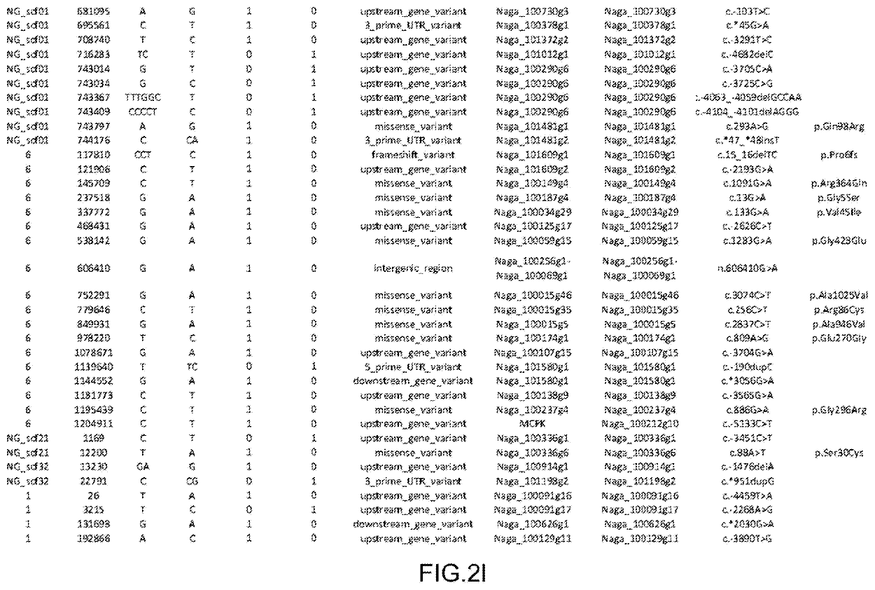

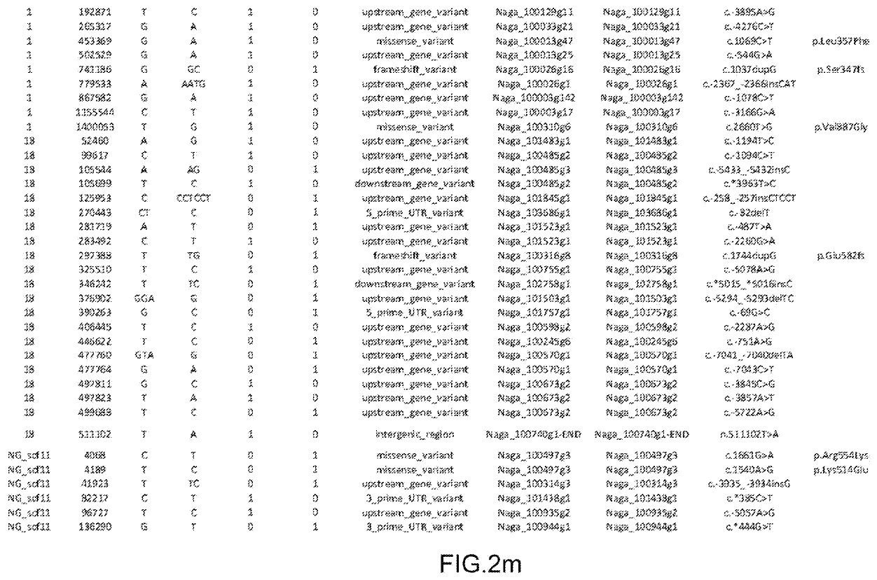

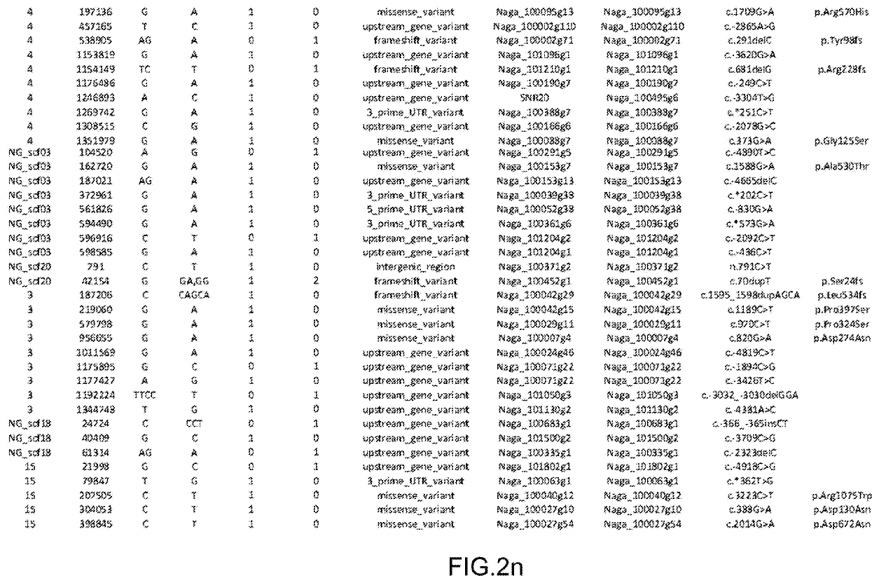

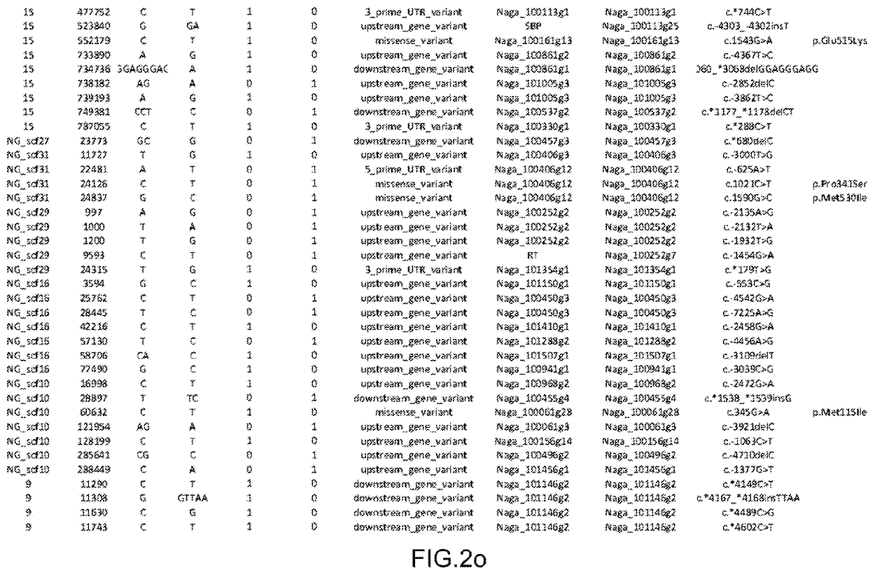

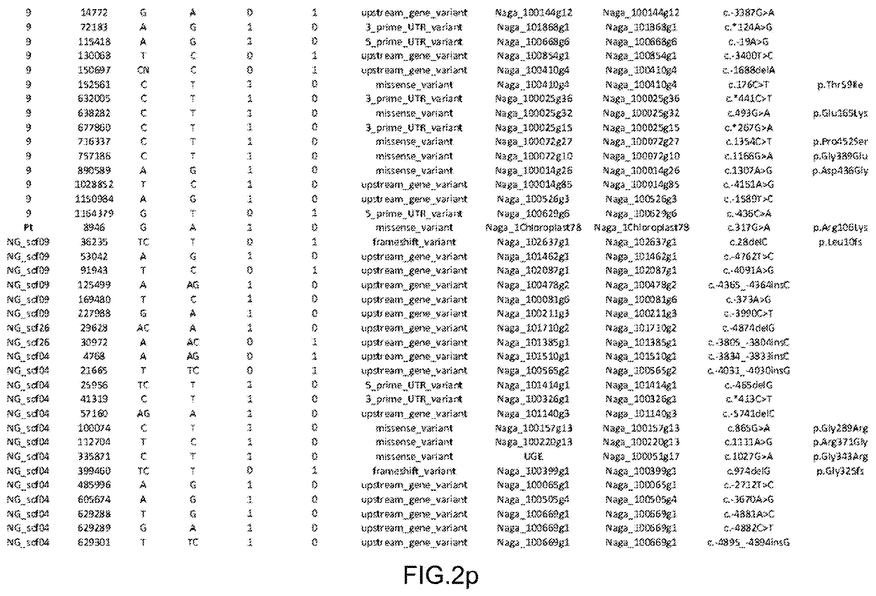

The list of mutations identified is shown in FIG. 2.

Among the 504 variants identified, a missense mutation (Naga_100050g23) on the carotenoid oxygenase enzyme could be responsible for an altered carotenoid biosynthetic pathway, thus leading to the increased production of Astaxanthin and Cantaxanthin as observed in this mutant with respect to the wild-type form.

Moreover, another missense mutation on the enzyme glutamate synthase (Naga_100005g23) suggests possible reduced activity for this key enzyme for nitrogen assimilation and chlorophyll biosynthesis (Gomez-Silva et al., Planta 1985), thus making this mutation likely responsible for the reduced chlorophyll content and increased lipid accumulation phenotype observed in the ASTAOMEGA mutant.

A mutation on the chloroplast RNA polymerase subunit (Naga_1Chloroplast7) was also identified; this mutation generated reduced chloroplast transcription, resulting in reduced accumulation of chlorophyll-binding subunits.

According to a third subject matter, this invention describes a process for the production of Astaxanthin, ketocarotenoids, and Omega-3 (EPA) using the mutated strain.

In particular, the ASIAOMEGA mutant strain may be grown in growth media suitable for the cultivation of marine algae, such as, for example, f/2 medium (Guillard, R. R. L. & Ryther, J. H. Studies of marine planktonic diatoms, I, Cyclotella nanna (Hustedt) and Detonula convervacea (Cleve). Can. J. Microbiol. (1962)) in closed (e.g., photobioreactors) or open (commonly referred to as open ponds or raceway ponds) culture systems.

Cultivation may also be conducted in saline waters.

Production may occur under different light conditions, at different temperatures, and at different CO₂ concentrations.

Said cultivation may then be conducted under one or more of the following conditions:
light conditions between 20-1000 μmol photons m⁻² s⁻¹,
temperature between about 20-35° C. and preferably 20-25° C.,
CO₂ concentrations up to 15% and preferably between 0.03%-3% (v/v).

CO₂ could also be made available directly in the growth medium, for example, in the form of carbonate.

Glucose, or another reduced carbon source such as glycerol or ethanol, may be added to the growth medium to improve productivity.

Cultivation may be conducted until the saturation phase is reached, such as in 4-8 days.

Longer cultivation in the saturation phase results in increased ketocarotenoid content.

Pigment analysis demonstrates the production of Astaxanthin, Cantaxanthin, and other trace ketocarotenoids.

The productivity values obtained averaged out to a productivity of:

| | | |
|---|---|---|
| 0.14-0.17 | g/L/day | biomass |
| 0.5-0.7 | mg/L/day | ASX |
| | | (and ketocarotenoids) |
| 2.97-4.54 | mg/L/day | EPA |

Glucose may increase biomass productivity by up to 0.22 g/L/day, but may reduce the percentage of ketocarotenoids, resulting in a ketocarotenoid productivity of 0.75 mg/L/day.

Astaxanthin and lipids may be extracted from the cells according to the same methodologies used to extract Astaxanthin produced by Hematococcus pluvialis.

According to a preferred aspect of this invention, the ASTAOMEGA strain may be grown under one or more of the following conditions:
in commercially available f/2 culture medium and preferably in photobioreactors with volumes ranging from 80 mL to 20 L;
by air insufflated from the bottom of the photobioreactor enriched with varying concentrations of CO₂, preferably between 300 ppm and 30,000 ppm. In any case, the enrichment of the insufflated air with CO₂ may be modulated based on the pH of the growth medium as an index of CO₂ consumption by the cultured micro algae.

The function of CO₂-enriched air insufflation is both to promote gas exchange in the culture medium, by supplying CO₂ and reducing the O₂ concentration so as to promote photosynthetic activity of the cells, and to prevent, or reduce, cell sedimentation.

Cultivation in photobioreactors is conducted for a variable cultivation time, preferably until the saturation phase is reached (3 to 10 days, preferably 4 to 8 days), achieving biomass, ketocarotenoid, Astaxanthin, and EPA production yields in line with the above.

According to a particular aspect of the invention, the culture of the ASTAOMEGA strain may also be carried out in the presence of an appropriate amount of a carbon source, such as, for example, glucose, preferably in an amount of about 0.5-40 g/L, or a similar amount of a reduced carbon source such as glycerol or ethanol, in order to improve productivity.

A number of illustrative variants briefly set forth below may be applied to the process conditions according to this invention.

The production may be done by considering one or more of the following variants:
in closed photobioreactors or in open systems (“open ponds” or “raceway ponds”), as well as in other devices developed for microalgae cultivation such as hybrid systems or biofilm cultivation systems;
with discontinuous (batch), semi-discontinuous (semi-batch), continuous or semi-continuous cultivation methods;
indoors or outdoors;
by means of LED lighting.

In addition, the mutated strain preparation of this invention may also be achieved by genome-specific editing of the N. gaditana genome by reproducing all or parts of the introduced mutations.

Thus, according to another subject matter of this invention, the same mutations of the ASTAOMEGA strain of this patent application are described to induce other microalgae species (marine and/or non-marine species) to produce Astaxanthin.

The ASTAOMEGA technology of this invention may be extended to all the different applications in which Astaxanthin is required or involved, including those in which Astaxanthin is a metabolic intermediate or by-product.

An automatic algae harvesting phase may also be integrated into the process to further reduce and optimize production costs.

Thus, the process of this invention enables the production of a mixture of astaxanthin and eicosapentaenoic acid.

More specifically, this mixture has an eicosapentaenoic acid/astaxanthin ratio by weight in the range of 4.4 to 7.9.

According to one particular aspect, the described process also allows an algal biomass to be obtained, which is rich in astaxanthin and eicosapentaenoic acid.

More specifically, this biomass has an eicosapentaenoic acid/astaxanthin weight ratio in the range of 4.4 to 7.9.

In a fourth subject matter, this invention describes food, pharmaceutical, nutraceutical, or cosmetic compositions comprising the mixture of compounds produced by the mutated strain.

According to other subject matters of this invention, the use of the compounds produced by the mutated strain in the food, pharmaceutical, nutraceutical, and cosmetic industries is described.

The compositions or formulations are achievable by the person skilled in the art by using the common technologies of pharmaceutical preparative technique known in the art, with the addition or not of the appropriate additives, carriers, excipients, and/or active ingredients, depending on the type of product and/or form of administration desired.

According to a particular aspect of this invention, the produced compounds find application in the aquaculture industry.

Said compounds are, in fact, responsible for fish pigmentation, which is recognized as a difficult quality trait to achieve in farm-raised fish.

The biomass obtained from the culture process may also be used in aquaculture and, in particular, as fish feed.

For this object, an automatic algae harvesting phase may be integrated into the process to further reduce and optimize production costs.

In this way, the oil enriched in Astaxanthin and EPA may be put to the most valuable uses, while the biomass remaining after oil extraction, which in each case is enriched in Astaxanthin and EPA, may be used as fish feed.

### Example

The ASTAOMEGA strain was grown in batch airborne photobioreactors under continuous white light at 500 μmol photons m⁻² s⁻¹ in F/2 medium. The device used for microalgae cultivation was the MC 1000-OD from PSI (Photon Systems Instruments) spol. s r.o. Drásov 470, 664 24 Drasov, Czech Republic. Air enriched with 3% CO₂ was bubbled from the bottom of the photobioreactors.
The composition of the F/2 soil was as follows: 0.092 g/L Guillard's (F/2), seawater enrichment solution (Merk G0154), 32 g/L sea salt (Merck S9883), TRIS-HCl 4.84 g/L, thiamine 0.1 mg/L, biotin 0.5 μg/L, vitamin B2 0.5 μg/L.
Growth was conducted for 5 days resulting at the end of growth in a total dry biomass of 0.87±0.02 g/L with a mean daily biomass productivity of 0.17±0.01 mg/L/day and a maximum daily biomass productivity of 0.35±0.01 mg/L/day. In this condition, total lipid productivity was 40.39±4.43 mg/L/day and EPA productivity was 3.22±0.31 mg/L/day. The productivity of ketocarotenoids is ±0.04 mg/L/day.
Under the same conditions, but with the addition of 10 g/L glucose to the culture medium, a total dry biomass of 1.03±0.14 g/L with a mean daily biomass productivity of 0.21±0.01 mg/L/day and a maximum daily biomass productivity of 0.39±0.06 mg/L/day. In this condition, total lipid productivity was 50.62±16.7 mg/L/day and EPA productivity was 3.67±1.16 mg/L/day. Ketocarotenoid productivity was 0.76±0.12 mg/L/day.

From the description provided above, the advantages provided by this invention will be apparent to the person skilled in the art.

In particular, with regard to the mutated strain, it allows for high amounts of astaxanthin and Omega-3 (in particular, EPA) to be obtained.

This allows for the preparation of numerous formulations enriched in Omega-3 and/or Astaxanthin.

Of the already known products, in fact, both Omega-3 and Astaxanthin are present only in krill oil, but Astaxanthin is in a concentration generally lower than 0.05%.

Given that Nannochloropsis algae has recently been proposed in Europe as a novel food for human consumption and is already approved by the FDA (FDA 2015; US Food and Drug Administration—New Dietary Ingredient Notification Report #826. http://www.regulations.gov/#!documentDetail;D=FDA-2014-S-0023-0041), the use of the ASTAOMEGA mutant strain appears to be an innovative solution.

The mutant strain ASTAOMEGA CCAP 849/16 has been identified as a non-GMO (Non-Genetically Modified Organism) and therefore its cultivation is allowed at industrial level without being subject to the restrictions required for GMOs.

The ASTAOMEGA mutant strain from N. gaditana W.T. is unexpectedly characterized by some unique features, including:
accumulation of Astaxanthin (up to 1% by weight (w/w) per dry weight of algae biomass) and simultaneously Omega-3 EPA fatty acid;
reduced heat dissipation, which is significant for maintaining efficient photosynthesis;
rapid growth of the species with no reduction in biomass production associated with Astaxanthin production;
reduced chlorophyll content, which allows for better light penetration into the photobioreactor (due to reduced pigmentation).

Regarding the process for producing astaxanthin, the following advantages may be pointed out:
increased productivity, due to the significant accumulation of Astaxanthin (up to 1% (w/w) of its dry weight) with respect to the starting strain Nannochloropsis gaditana wild-type, in which Astaxanthin is normally produced only in trace amounts;
reduction of production costs, based both on the higher productivity of the ASTAOMEGA strain;
reduction of production costs, based on the elimination of the stress phase (“red” phase), and the possibility of accumulating Astaxanthin;
increased environmental sustainability, both through the elimination of the stress phase mentioned above (which requires a lot of energy in order to provide intense light and high temperature) increased light intensity and temperature) for Astaxanthin production.

The resulting preparations based on the compounds of the invention further enable compositions and formulations to be offered for human use in the nutraceutical, pharmaceutical, and cosmetic industries.

The use of the compounds of the invention in the aquaculture industry, on the other hand, gives fish farmers the opportunity to improve fish quality by increasing both pigmentation and Omega-3 content.

What is claimed is:

1. The mutant algal strain ASTAOMEGA (formerly Nannochloropsis gaditana D23) deposited in the Culture Collection of Algae and Protozoa (CCAP) (SAMS Limited Scottish Marine Institute; OBAN, Argyll, PA37 1QA, UK) on 28 Jan. 2016 under the Accession Number given by the International Depositary Authority CCAP 849/16.

2. The mutant algal strain of claim 1, characterized by 504 mutations as illustrated in FIG. 2.

3. A method for obtaining the mutant algal strain ASTAOMEGA (formerly Nannochloropsis gaditana D23) deposited in the Culture Collection of Algae and Protozoa (CCAP) (SAMS Limited Scottish Marine Institute; OBAN, Argyll, PA37 1QA, UK) on 28 Jan. 2016 under the Accession Number given by the International Depositary Authority CCAP 849/16, the method comprising subjecting the alga Nannochloropsis gaditana W.T. to a random chemical mutation phase by exposure to the mutagenic agent ethyl methanesulfonate.

4. The method of claim 3, wherein said exposure to the mutagenic agent ethyl methanesulfonate at a final weight/volume percentage is of about 2%.

5. The method of claim 3, wherein said exposure is protracted for a time of 2 hours.

6. A process for producing astaxanthin and omega-3 fatty acids, particularly eicosapentaenoic acid, the process comprising culturing the mutant algal strain of claim 1.

7. The process of claim 6, wherein said cultivation is conducted in the presence of glucose, glycerol, or ethanol.

8. The process of claim 7, wherein said cultivation is conducted in the presence of glucose in an amount of about 1.5-40 g/L.

9. The process of claim 6, wherein said cultivation is conducted employing a light emitting white light varying from 20 to 1000 μmol photons m⁻² s⁻¹.

10. The process of claim 6, wherein said cultivation is conducted at a temperature between about 20° C. and 35° C.

11. The process of claim 6, wherein said cultivation is conducted in the presence of up to about 15% CO₂.

12. The process of claim 6, wherein said process further produces an algal biomass.

13. A mixture of astaxanthin and eicosapentaenoic acid obtained according to the process of claim 6.

14. The mixture of claim 13, comprising an eicosapentaenoic acid/astaxanthin ratio of about 4.4-7.9.

15. An algal biomass obtained according to the process of claim 12.

16. A food, pharmaceutical, nutraceutical or cosmetic composition or a zootechnical supplement or feed comprising the mixture of astaxanthin and eicosapentaenoic acid of claim 13.

17. A method for producing astaxanthin and omega-3 eicosapentaenoic acid (EPA), the method comprising using the mutant algal strain of claim 1.

18. The mixture of astaxanthin and eicosapentaenoic acid of claim 13, wherein the mixture is used in food, pharmaceutical, nutraceutical, or cosmetic products.

19. The mixture of astaxanthin and eicosapentaenoic acid of claim 13, wherein the mixture is used in aquaculture.

20. The algal biomass of claim 12, wherein the algal biomass is used as feed.
